(19) United States
(12) Patent Application Publication
(10) Pub. No.: US 2016/0101977 A1
(43) Pub. Date: Apr. 14, 2016

(54) METHOD AND APPARATUS FOR CAPTURING AND SEQUESTERING CARBON

(71) Applicant: BE ENERGY GROUP, INC., ST. CATHARINES (CA)

(72) Inventors: Donald GUILIAN, Chagrin Falls, OH (US); Fred HAEMER, Pueblo, CO (US)

(73) Assignee: BE ENERGY GROUP, INC., ST. CATHARINES (CA)

(21) Appl. No.: 14/879,813

(22) Filed: Oct. 9, 2015

Related U.S. Application Data
(60) Provisional application No. 62/062,058, filed on Oct. 9, 2014.

(51) Int. Cl. C01B 3/44; C07C 1/24; C07C 2/86; C07C 51/00; C01B 33/12

(52) U.S. Cl. CPC C01B 3/44; C07C 51/00; C01B 33/128; C07C 2/862; C07C 1/24; C01B 2203/86; C01B 2203/0238; C01B 2203/1241

(57) ABSTRACT

A method for converting carbon dioxide (CO2) into useful carbonaceous compounds includes the steps of recovering CO2 from a CO2 emitting source, passing the recovered CO2 through a CO2 stripper, and using CO2 passed through the CO2 stripper as feedstock for reactions that generate useful carbonaceous compounds. The method further reduces CO2 emissions by preparing CO2 to be used as feedstock to drive other beneficial reactions.

### CROSS REFERENCE TO RELATED APPLICATIONS

This application claims the benefit of U.S. provisional application No. 62/062,058, filed Oct. 9, 2014, the contents of which are incorporated herein by reference.

### FIELD OF INVENTION

The present application relates to synthetic fuels, and more specifically to a method for converting carbon dioxide to synthetic fuels and useful derivatives.

### BACKGROUND

Power plants consume fossil fuel to produce electricity. The power plant effluent is carbon dioxide, water, nitrogen oxide, sulfur oxide and heavy metals. Total man-made carbon dioxide (CO2) emissions in the world today amount to approximately 30 giga-tons per year, with fossil fuel-based power stations contributing in excess of 30% of this total.

Carbon dioxide separation is a commercial process, but power companies are not able to recoup the costs of the recovery.

Schemes for the sequestration of carbon dioxide and subsequent conversion require the same costly CO2 Compression and infrastructure for CO2 Storage.

Oil refineries can convert CO2 into synthesis gas and derivatives, but the size of oil refineries is too great for a typical oil refinery to be located at power plants.

### SUMMARY

There is a need in the industry for a system and process for efficiently utilizing CO2 emissions for beneficial reactions and to reduce CO2 emissions into the atmosphere.

The present invention solves the foregoing problems by using existing commercial processes in a novel manner that will convert power plant emissions into useful products at a competitive price and with no harm to the environment. According to one of many embodiments of the invention, carbon dioxide emissions can be converted and provide up to 25% of the carbon in the useful product of transportation fuel. According to another embodiment, carbon dioxide can be converted to provide up to 50% of the useful product of acetic acid, which is widely used in the production textiles.

According to another embodiment of the invention, a dry reformer can use carbon dioxide and methane as the feedstock with a level of carbon dioxide at 50% by weight. An enhanced catalyst can be employed to produce a synthesis gas.

A Thermochem Recovery International (TRI) reformer is a pulsed fluidized bed. According to another embodiment of the invention, more reactive catalysts can be used in the TRI reformer than are normally used. Hydrogen also can be used according to several aspects of the invention. Hydrogen is required downstream in processes used to make methanol, dimethyl ether and gasoline. Because of the feasibility for using hydrogen, a hydrogen generation system can be included. The TRI reformer can also accommodate the use of powdered carbon and powdered iron as catalysts.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a block diagram that shows the process of converting carbon dioxide to gasoline; and

FIG. 2 is a block diagram that shows the process of converting carbon dioxide to mineral carbonates.

### EMBODIMENTS OF THE INVENTION

One aspect of the invention is a method for converting carbon dioxide (CO2) into useful carbonaceous compounds by recovering CO2 from a CO2 emitting source; passing the recovered CO2 through a CO2 stripper; and using CO2 passed through the CO2 stripper as feedstock for reactions that generate useful carbonaceous compounds.

A second aspect of the invention is a method for producing gasoline by diverting CO2 from an electric generating unit to an amine scrubber to generate a CO2-rich amine absorber fluid; flowing the CO2-rich amine absorber fluid to a dry reformer; and mixing the CO2-rich amine absorber fluid in the dry reformer with methane and steam to yield a synthesis gas.

A third aspect of the invention is a method for fractionating olivine by diverting CO2 from an electric generating unit to an amine scrubber to generate a CO2-rich amine absorber fluid; flowing the CO2-rich amine absorber fluid to a carbon dioxide stripper; and contacting a solution of soluble silicic acid with the CO2-rich amine absorber fluid to form carbonic acid, wherein the carbonic acid precipitates silica.

### EXAMPLES

### Example 1

An embodiment of the present invention will enable the use of more reactive catalysts in a pulsed fluidized bed reformer, such as the pulsed fluidized bed reformer manufactured commercially by Thermochem Recovery International (TRI). For example:

1.30CH₄+H₂O+0.30CO₂→1.60CO+3.60H₂ (Equation 1)

Giving a Hydrogen to Carbon ratio (H₂/C) of 2.25

Another aspect of the invention can accommodate the use of hydrogen. Hydrogen is required downstream in processes used to make methanol, dimethyl ether and gasoline. A carbon dioxide refinery contemplated herein will have a requirement for hydrogen that can justify a hydrogen generation system.

0.6H₂+0.6CO₂+1CH₄+0.4H₂O→1.6CO+3.6H₂ (Equation 2)

A TRI reformer also can accommodate the use of powdered carbon and powdered iron as catalysts.

1CH₄+1H₂O→1CO+3H₂ (Equation 3a)

0.166Fe+0.166CO₂→0.166FeO+0.166CO (Equation 3b)

0.166C+0.166FeO→0.166Fe+0.166CO (Equation 3c)

Net reaction

1CH₄+1H₂O+0.166CO₂+0.166C→1.33CO+3H₂ (Equation 4)

Giving a H₂/C of 2.25

### Example 2

A series of catalysts can be used to recover CO2 from vent gas from the direct reduction of iron during the process of converting iron ore into cast iron.

Dry reforming recycle gas

4H2+2CO2+2CH4→4CO+8H2 (Equation 5)

Direct reduction of Iron

1Fe2O3+2CO+4H2→2Fe+4H2O+2CO2 (Equation 6)

A commercial reformer and nickel catalysts can be used to convert carbon dioxide emissions into synthesis gas. The main process uses synthesis gas to reduce the iron ore. The process for making iron produces flue gas that contains carbon dioxide. The flue gas can be recycled into a reformer. The flue gas and natural gas can be converted into synthesis gas for the iron ore process.

### Example 3

A fluidized bed dry reformer, e.g., such as that manufactured by Thermochem Recovery International, can be used for the converting carbon dioxide and natural gas into synthesis gas to produce derivatives such as gasoline, alcohols, acids, glycols, monomers, polymers and other petrochemical products.

Feedstock: natural gas/CO2.

Feedstock ratio: 1/1 (weight)

Yield: When fed to the Primus Green Multistep single recycle MTG process: 7.25 gallons premium gasoline per MCF of natural gas. When fed to a single step DME hydrocarbon synthesis: 7.9 gallons premium gasoline per MCF of natural gas.

Carbon dioxide to gasoline

5.75CH₄+5.75CO₂→11.5CO+11.5H₂ (Equation 7a)

0.5CH₄+0.5H₂O→0.5CO+1.5H₂ (Equation 7b)

12CO+13H₂→4CH₃OCH₃+4CO₂ (Equation 7c)

4CH₃OCH₃→1C₈H₁₈+4H₂O (Equation 7d)

Overall reaction

6.25CH₄+1.75CO₂→1C₈H₁₈+3.5H₂O (Equation 8)

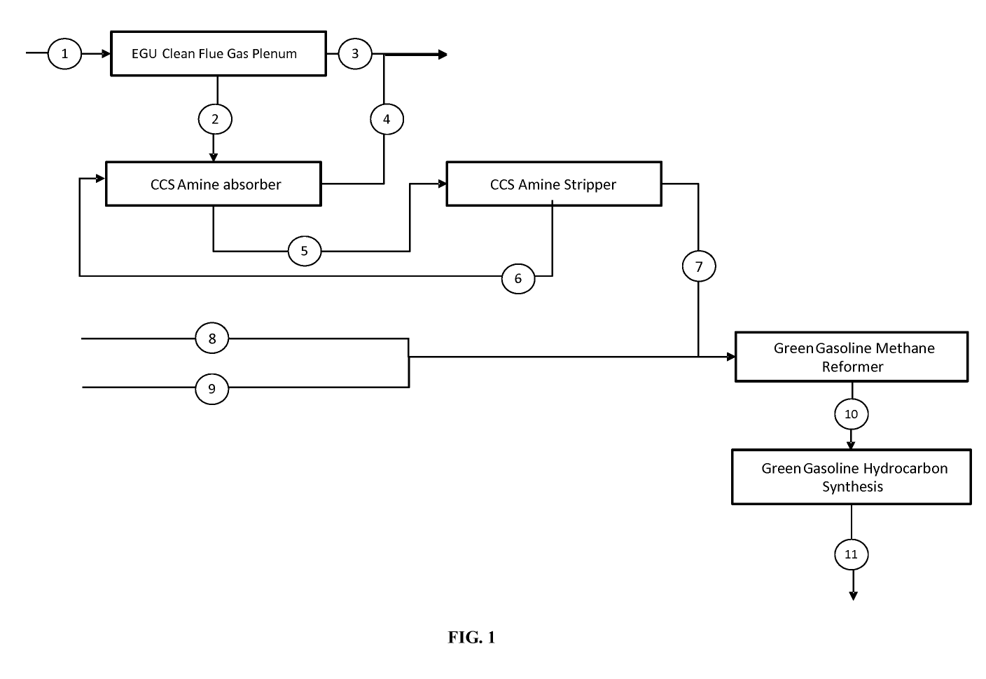

Referring generally to FIG. 1, an electric generating unit (EGU) operating on coal can have two furnaces receiving a feed of air and pulverized coal. Nearly complete combustion of the coal and oxygen is achieved in the furnace. The combustion gas is cooled by a steam boiler.

The combustion gases are treated by a sulfur recovery scrubber. Carbon black can be injected to remove mercury; and a bag house can be used to remove particulates. The stack gas is discharged to a vent stack (Stream One). A portion of the vent gas (Stream Two) is directed to an amine scrubber. The balance of the vent gas (Steam Three) continues to the vent stack.

In the amine scrubber CO2 is removed from the vent gas. The lean vent gas (Stream Four) is directed to the vent stack. The CO2 rich amine absorber fluid (Stream Five) is directed to a carbon dioxide stripper. Unlike the present invention, most other CO2 separation technologies rely on a cold fluid to absorb CO2 and a heater to strip CO2. The CO2 lean fluid is returned to the absorber (Stream Six). The stripped CO2 (Stream Seven) is appropriate for consumption in a dry reformer of methane (Steam Eight). When combined with steam (Stream Nine) reforming of methane, a synthesis gas (Stream Ten) appropriate for production of hydrocarbons is obtained. Gasoline (Stream Eleven) can be formed in a methanol to gasoline synthesis.

In the hypothetical application to an example facility the disclosure is illustrated further. The example EGU is a coal fired steam turbine cycle. The rated capacity is 344 Mw. Before application stack gas flows at 310000 scfm to the atmosphere emitting 60,000 scfm of carbon dioxide. In applying the technology 10000 scfm are diverted to the absorber. From the absorber 8000 scfm of vent gas carrying no CO2 are returned to the stack flow. The stack now emits 58,000 scfm of CO2. From the stripper 2000 scfm of CO2 flow to a dry reformer where it mixes with 7200 scfm of methane and 340 lb/min steam in a reaction producing a synthesis gas with a hydrogen to carbon ratio of 2.35. The synthesis gas is consumed in a hydrocarbon synthesis to produce 2000 barrels per day of gasoline. This 3% reduction in CO2 emissions contributes to the statewide GHG emission reduction required until 2023. The application of a second module in 2023 further contributes to the compliance. The application of a third module in 2027 contributes to the compliance schedule. Application of a 4th module in 2030 contributes to completing the compliance cycle for this facility.

Referring to FIG. 1, the streams are identified as follows:

ID Description

1 Stack Gas from an EGU

2 Diverted Stack Gas

3 Remaining stack gas

4 Lean (no CO2) Gas Returned to Stack

5 Rich absorber liquid

6 Weak absorber liquid

7 Carbon dioxide to dry reforming

8 Purchased methane

9 Steam from Gasoline synthesis heat recovery

10 Synthesis Gas

11 Gasoline for market.

### Example 4

A fourth example is the application of carbon dioxide oxidative coupling of methane to produce ethylene. Recent breakthroughs in catalysis allow the production of ethylene from carbon dioxide and methane. While ethylene has inherent commercial value, the use of catalytic condensation can produce targeted alkane hydrocarbons such as diesel fuel. After ethylene is separated from the water from the coupling reaction, half of the ethylene is reacted with additional methane by catalytic condensation to yield a propane rich stream. In a second catalytic condensation step, the remaining ethylene is reacted with the propane rich stream to yield a pentane rich steam.

Carbon dioxide to ethylene

1.5CH₄+0.5CO₂→1C₂H₄+1H₂O (Equation 9a)

Ethylene to Propane

1CH₄+1C₂H₄→1C₃H₈ (Equation 9b)

Ethylene to Pentane

1C₂H₄+1C₃H₈→C₅H₁₂ (Equation 9c)

### Example 5

A fifth example is again the use of Carbon Dioxide oxidative coupling of methane to produce ethylene. The inherent value of the ethylene is improved by the use of ethylene oligomerization to produce linear alpha olefins in the C6 to C18 range.

### Example 6

A sixth example is the production of acetic acid.

### Example 7

A seventh example is the refining of olivine (a silicate mineral). In this process carbon dioxide is captured as described in example three, however the carbon dioxide is applied to a slurry of olivine producing finely divided magnesite (MgCO₃) useful as a pigment and an aqueous solution of silicic acid. In a second step the filtrate is further contacted with carbon dioxide. The carbonic acid thus formed precipitates a finely divided silica also useful as a pigment.

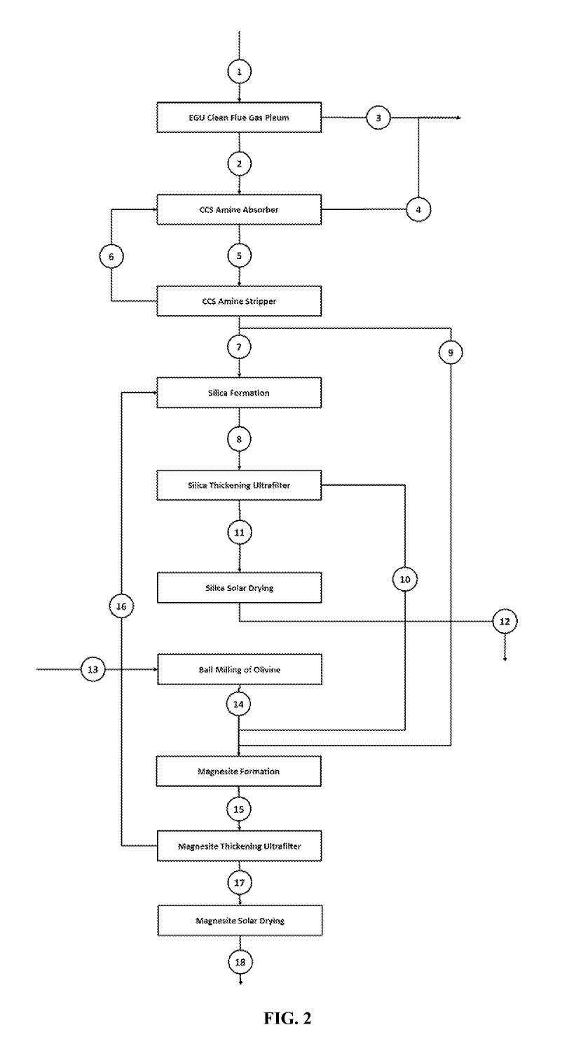

More specifically, as shown in FIG. 2 an EGU operating on coal can have two furnaces receiving a feed of air and pulverized coal. Nearly complete combustion of the coal and oxygen is achieved in the furnace. The combustion gas is cooled by the steam boiler.

The combustion gases are treated by a sulfur recovery scrubber Carbon black can be injected to remove mercury, and a bag house is used to remove particulates. As shown in FIG. 1, the stack gas is discharged to a vent stack (Stream One). A portion of the vent gas (Stream Two) is directed to an amine scrubber. The balance of the vent gas (Steam Three) continues to the vent stack.

In the amine scrubber CO2 is removed from the vent gas. The lean vent gas (Stream Four) is directed to the vent stack. The CO2 rich amine absorber fluid (Stream Five) is directed to a carbon dioxide stripper. (Most CO2 separation technologies rely on a cold fluid to absorb CO2 and a heater to strip CO2). The CO2 lean fluid is returned to the absorber (Stream Six).

The CO2 is applied in a mass integration scheme to fractionate the olivine mineral. The stripped CO2 (Streams Seven and Nine) are appropriate for consumption in an olivine mineral refining process to yield pigments. A solution of soluble silicic acid (Steam Sixteen) is contacted with carbon dioxide (Stream Seven), the carbonic acid formed precipitates silica. The resulting slurry (Stream Eight) is thickened by ultrafiltration. The filtrate (Stream Ten) is fed to the next step. The retentate (Stream Eleven) is feed to a solar dryer. The dried solids (Stream Twelve) are a salable pigment.

Precipitation of Silica from silicate solution

2CO₂+2H₂O→4H⁺+2CO₃⁻² (Equation 10a)

SiO₄⁻⁴+4H⁺→SiO₂+2H₂O (Equation 10b)

In the second step of processing, raw olivine (Stream Thirteen) is ball milled to a finely divided powder and slurried (Stream Fourteen). The slurry is contacted with filtrate (Stream Ten) and carbon dioxide (Stream Nine). In this step, finely divided magnesite is formed and silicate in the mineral is solubilized, the slurry (Stream Fifteen) is thickened by ultrafiltration. The silica rich filtrate (Stream Sixteen) is fed to the first processing step described above. The magnesium rich retentate (Stream Seventeen) is sent to a solar dryer. The resulting powder (Stream Eighteen) is a salable pigment.

Carbonation of Olivine

MgSiO₂+CO₂+3H₂O→MgCO₃+SiO₄⁻⁴+4H+ (Equation 11)

Referring to FIG. 2, the following streams are identified:

ID Description

1 Stack Gas from an EGU

2 Diverted Stack Gas

3 Remaining stack gas

4 Lean (no CO2) Gas Returned to Stack

5 Rich absorber liquid

6 Weak absorber liquid

7 Carbon Dioxide to Silica Formation

8 Silica Slurry to Thickening

9 Carbon Dioxide to Magnesite Formation

10 Lean Filtrate to Magnesite Formation

11 Thickened Silica to Solar Drying

12 Silica Pigment to Market

13 Olivine to Ball Mill

14 Olivine Slurry to Magnesite Formation

15 Magnesite Slurry to Thickening

16 Silica rich Filtrate to Silica Formation

17 Thickened Magnesite to Solar Drying

18 Magnesite Pigment to Market

### CONCLUSION

While various preferred embodiments have been shown and described, modifications thereof can be made by one skilled in the art without departing from the spirit and teachings herein. The embodiments herein are exemplary only, and are not limiting. Many variations and modifications of the apparatus disclosed herein are possible and within the scope of the invention. Accordingly, the scope of protection is not limited by the description set out above.

What is claimed is:

1. A method for converting carbon dioxide (CO2) into useful carbonaceous compounds, comprising:
recovering CO2 from a CO2 emitting source;
passing the recovered CO2 through a CO2 stripper; and
using CO2 passed through the CO2 stripper as feedstock for reactions that generate useful carbonaceous compounds.

2. The method of claim 1, wherein the CO2 emitting source is an electric generating unit (EGU).

3. The method of claim 1, wherein CO2 is recovered by passing vent gas from an electric generating unit through an amine scrubber.

4. The method of claim 3, wherein the recovered CO2 is a vapor.

5. The method of claim 4, wherein the CO2 vapor further comprises water vapor.

6. The method of claim 1, wherein the useful carbonaceous compounds are synthesis gas, gasoline, methanol, ethylene, alkane hydrocarbons, acetic acid, magnesite, silicic acid, or silica.

7. The method of claim 6, wherein the alkane hydrocarbons are diesel fuel.

8. A method for producing gasoline, comprising:
diverting CO2 from an electric generating unit to an amine scrubber to generate a CO2-rich amine absorber fluid;
flowing the CO2-rich amine absorber fluid to a dry reformer; and
mixing the CO2-rich amine absorber fluid in the dry reformer with methane and steam to yield a synthesis gas.

9. The method of claim 8, wherein the synthesis gas has a hydrogen to carbon ratio of about 2 to about 4.

10. The method of claim 8, wherein the synthesis gas has a hydrogen to carbon ratio of about 2 to about 3.

11. The method of claim 8, wherein the synthesis gas has a hydrogen to carbon ratio of about 2.35.

12. A method for fractionating olivine, comprising:
diverting CO2 from an electric generating unit to an amine scrubber to generate a CO2-rich amine absorber fluid;
flowing the CO2-rich amine absorber fluid to a carbon dioxide stripper; and
contacting a solution of soluble silicic acid with the CO2-rich amine absorber fluid to form carbonic acid, wherein the carbonic acid precipitates silica.
